(19) United States
(12) Patent Application Publication
(10) Pub. No.: US 2010/0249591 A1
(43) Pub. Date: Sep. 30, 2010

(54) SYSTEM AND METHOD FOR DISPLAYING ULTRASOUND MOTION TRACKING INFORMATION

(76) Inventors: Andreas Heimdal, Oslo (NO); Stian Langeland, Stavern (NO); Fredrik Orderud, Trondheim (NO)

Correspondence Address: DEAN D. SMALL;THE SMALL PATENT LAW GROUP LLP, 225 S. MERAMEC, STE. 725T, ST. LOUIS, MO, 63105, US

(21) Appl. No.: 12/410,421

(22) Filed: Mar. 24, 2009

(51) Int. Cl. A61B 8/14; G06K 9/00

(52) U.S. Cl. 600/443; 382/128

(57) ABSTRACT

A system and method for displaying ultrasound motion tracking information are provided. The method includes obtaining three-dimensional (3D) ultrasound image data of a scanned object. The 3D ultrasound image data includes motion tracking information. The method further includes transforming the 3D ultrasound image data with the motion tracking information to a two-dimensional (2D) map projection and generating a 2D map based on the 2D map projection.

### BACKGROUND OF THE INVENTION

This invention relates generally to diagnostic imaging systems, and more particularly, to ultrasound imaging systems providing motion tracking, especially for cardiac imaging.

Medical imaging systems are used in different applications to image different regions or areas (e.g., different organs) of patients. For example, ultrasound imaging systems are finding use in an increasing number of applications, such as to generate images of the heart. In heart imaging applications, motion tracking of the muscles of the heart based on acquired ultrasound images of the heart also may be provided using, for example, two-dimensional (2D) or three-dimensional (3D) speckle tracking. Speckle tracking uses speckle information in the acquired images to track motion, such as motion of the myocardium of the imaged heart. These images are then displayed for review and analysis by a user, which may include 2D strain analysis of myocardial deformation.

In order to ensure that the tracking was performed properly, a user typically reviews a display showing tracking information, which may include a graphical overlay. For example, some known ultrasound systems that provide motion tracking information use a tracked centerline of the imaged heart. For example, when performing cardiac image motion tracking in these known systems, the user thereafter compares the relative motion of the imaged heart and an overlay representing the tracked motion. However, because the error in tracking is typically much smaller than the muscle motion of the heart, it is often difficult to determine whether the tracking is correct, and if incorrect, where exactly the motion tracking failed. Additionally, the muscle motion is also fast, thereby making it difficult to follow the overlay, especially in the early relaxation stage of the heart. Accordingly, using known ultrasound systems displaying tracking information, it is often very difficult to visually confirm motion tracking results, for example, because the markings provided as part of the overlay move too quickly or too much to correlate with the motion of the heart. Thus, users may improperly confirm tracked motion.

Additionally, some known systems display a modified curved anatomical grayscale M-mode based on the tracked centerline. In such a display, the grayscale pattern will appear as horizontal lines if the tracking works correctly, and as non-straight lines if not working correctly. At least one disadvantage of this type of display is that an M-mode with horizontal lines normally indicates abnormal function. Users may react improperly or incorrectly to the abnormal display.

### BRIEF DESCRIPTION OF THE INVENTION

In accordance with an embodiment of the invention, a method for providing ultrasound information includes obtaining three-dimensional (3D) ultrasound image data of a scanned object. The 3D ultrasound image data includes motion tracking information. The method further includes transforming the 3D ultrasound image data with the motion tracking information to a two-dimensional (2D) map projection and generating a 2D map based on the 2D map projection.

In accordance with another embodiment of the invention, a user interface is provided that includes a two-dimensional (2D) map portion corresponding to a tracked surface model of a three-dimensional (3D) ultrasound imaged object. The user interface further includes a tracked motion display portion within the 2D map portion displaying grayscale motion information representative of tracked motion of the imaged object based on the tracked surface model.

In accordance with yet another embodiment of the invention, an ultrasound imaging system is provided that includes an ultrasound probe configured to acquire three-dimensional (3D) images of an object. The ultrasound system further includes a processor having a motion tracking module configured to generate a two-dimensional (2D) map projection based on grayscale motion data from the 3D images.

### BRIEF DESCRIPTION OF THE DRAWINGS

FIG. 1 is a block diagram of a diagnostic ultrasound imaging system configured to perform motion tracking and display motion tracking information in accordance with various embodiments of the invention.

FIG. 2 is a block diagram of an ultrasound processor module of the diagnostic ultrasound imaging system of FIG. 1 formed in accordance with various embodiments of the invention.

FIG. 3 is a flowchart of method for generating tracking information from three-dimensional (3D) ultrasound data in accordance with various embodiments of the invention.

FIG. 4 is a diagram illustrating transforming 3D ultrasound data having tracking information to a two-dimensional (2D) map based on a tracked surface model in accordance with various embodiments of the invention.

FIG. 5 is a diagram illustrating projection of tracking information for a tracked surface model to a rectangular 2D map in accordance with various embodiments of the invention.

FIG. 6 is a diagram illustrating projection of tracking information for a tracked surface model to a polar 2D map in accordance with various embodiments of the invention.

FIG. 7 is a diagram illustrating projection of tracking information for a tracked surface model to a semi-circle 2D map in accordance with various embodiments of the invention.

FIG. 8 is a display having a user interface displaying tracking information in a rectangular 2D map in accordance with various embodiments of the invention.

FIG. 9 is a display having a user interface displaying tracking information in a polar 2D map and illustrating motion in accordance with various embodiments of the invention.

FIG. 10 is a diagram illustrating segmented 2D maps corresponding to a segmented tracked surface model in accordance with various embodiments of the invention.

FIG. 11 is a diagram illustrating a 3D capable miniaturized ultrasound system formed in accordance with an embodiment of the invention.

FIG. 12 is a diagram illustrating a 3D capable hand carried or pocket-sized ultrasound imaging system formed in accordance with an embodiment of the invention.

FIG. 13 is a diagram illustrating a 3D capable console type ultrasound imaging system formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Exemplary embodiments of ultrasound imaging systems and methods for tracking motion and displaying tracked motion information are described in detail below. In particular, a detailed description of an exemplary ultrasound imaging system will first be provided followed by a detailed description of various embodiments of methods and systems for generating and displaying ultrasound motion tracking information, especially cardiac motion tracking information.

At least one technical effect of the various embodiments of the systems and methods described herein include generating a two-dimensional (2D) projection of three-dimensional (3D) ultrasound data for display as a map of grayscale data. In cardiac applications, a user then can confirm motion tracking information by viewing motion data in an easier configuration that does not look like a deformed heart and that shows little or no motion when tracking is good, namely that tracking quality is good. Accordingly, a user can more easily observe and determine which segments of the 2D projection have poor or less than acceptable lateral and circumferential tracking as the displayed grayscale pattern will appear to move in the lateral and/or circumferential direction. Moreover, a user is able to provide an input indicating where and how the tracking failed.

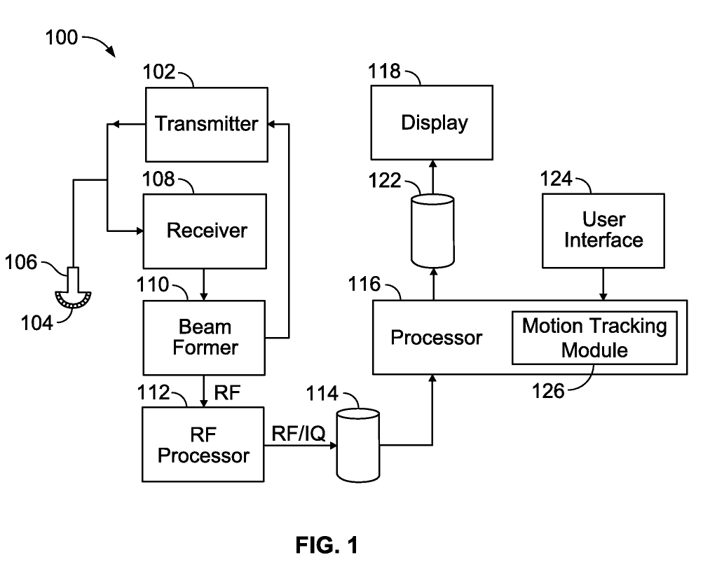

FIG. 1 is a block diagram of an ultrasound system 100 constructed in accordance with various embodiments of the invention. The ultrasound system 100 is capable of steering (mechanically and/or electronically) a soundbeam in 3D space, and is configurable to acquire information corresponding to a plurality of two-dimensional (2D) or three-dimensional (3D) representations or images of a region of interest (ROI) in a subject or patient. One such ROI may be a human heart or the myocardium (muscles) of a human heart. The ultrasound system 100 is also configurable to acquire 2D and 3D images in one or more planes of orientation. In operation, real-time ultrasound imaging using a matrix or 3D ultrasound probe may be provided.

The ultrasound system 100 includes a transmitter 102 that, under the guidance of a beamformer 110, drives an array of elements 104 (e.g., piezoelectric elements) within a probe 106 to emit pulsed ultrasonic signals into a body. A variety of geometries may be used. The ultrasonic signals are back-scattered from structures in the body, like blood cells or muscular tissue, to produce echoes that return to the elements 104. The echoes are received by a receiver 108. The received echoes are passed through the beamformer 110, which performs receive beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to a memory 114 for storage.

In the above-described embodiment, the beamformer 110 operates as a transmit and receive beamformer. In an alternative embodiment, the probe 106 includes a 2D array with sub-aperture receive beamforming inside the probe. The beamformer 110 may delay, apodize and sum each electrical signal with other electrical signals received from the probe 106. The summed signals represent echoes from the ultrasound beams or lines. The summed signals are output from the beamformer 110 to an RF processor 112. The RF processor 112 may generate different data types, such as B-mode, color Doppler (velocity/power/variance), tissue Doppler (velocity), and Doppler energy, for one or more scan planes or different scanning patterns. For example, the RF processor 112 may generate tissue Doppler data for multiple (e.g., three) scan planes. The RF processor 112 gathers the information (e.g. I/Q, B-mode, color Doppler, tissue Doppler, and Doppler energy information) related to multiple data slices and stores the data information with time stamp and orientation/rotation information in an image buffer 114.

The ultrasound system 100 also includes a processor 116 to process the acquired ultrasound information (e.g., RF signal data or IQ data pairs) and prepare frames of ultrasound information, which may include motion tracking information, for display on a display 118. The processor 116 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in memory 114 during a scanning session and then processed and displayed in an off-line operation.

The processor 116 is connected to a user interface 124 that may control operation of the processor 116 and receive user inputs as explained below in more detail. The user interface 124 may include hardware components (e.g., keyboard, mouse, trackball, etc.), software components (e.g., a user display) or a combination thereof. The processor 116 also includes a motion tracking module 126 that performs motion tracking and generates motion tracking information for display, which in some embodiments is displayed as a 2D projection map having a grayscale pattern.

The display 118 includes one or more monitors that present patient information, including diagnostic ultrasound images to the user for diagnosis and analysis (e.g., images with motion tracking information). One or both of memory 114 and memory 122 may store 3D data sets of the ultrasound data, where such 3D data sets are accessed to present 2D (and/or 3D images) as described herein. The images may be modified and the display settings of the display 118 also manually adjusted using the user interface 124.

It should be noted that although the various embodiments may be described in connection with an ultrasound system, the methods and systems described herein are not limited to ultrasound imaging or a particular configuration thereof. In particular, the various embodiments may be implemented in connection with different types of imaging, including, for example, magnetic resonance imaging (MRI) and computed-tomography (CT) imaging or combined imaging systems. Further, the various embodiments may be implemented in other non-medical imaging systems, for example, non-destructive testing systems.

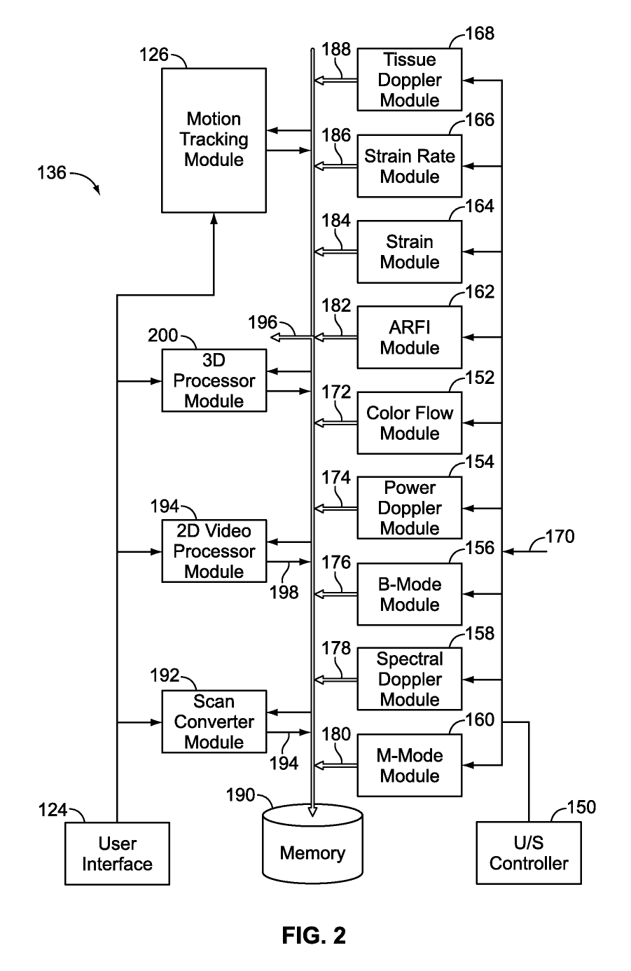

FIG. 2 illustrates an exemplary block diagram of an ultrasound processor module 136, which may be embodied as the processor 116 of FIG. 1 or a portion thereof. The ultrasound processor module 136 is illustrated conceptually as a collection of sub-modules, but may be implemented utilizing any combination of dedicated hardware boards, DSPs, processors, etc. Alternatively, the sub-modules of FIG. 2 may be implemented utilizing an off-the-shelf PC with a single processor or multiple processors, with the functional operations distributed between the processors. As a further option, the sub-modules of FIG. 2 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 2 may be controlled by a local ultrasound controller 150 or by the processor module 136. The sub-modules 152-168 perform mid-processor operations. The ultrasound processor module 136 may receive ultrasound data 170 in one of several forms. In the embodiment of FIG. 2, the received ultrasound data 170 constitutes I,Q data pairs representing the real and imaginary components associated with each data sample. The I,Q data pairs are provided to one or more of a color-flow sub-module 152, a power Doppler sub-module 154, a B-mode sub-module 156, a spectral Doppler sub-module 158 and an M-mode sub-module 160. Optionally, other sub-modules may be included such as an Acoustic Radiation Force Impulse (ARFI) sub-module 162, a strain module 164, a strain rate sub-module 166, a Tissue Doppler (TDE) sub-module 168, among others. The strain sub-module 162, strain rate sub-module 166 and TDE sub-module 168 together may define an echocardiographic processing portion.

Each of sub-modules 152-168 are configured to process the I,Q data pairs in a corresponding manner to generate color-flow data 172, power Doppler data 174, B-mode data 176, spectral Doppler data 178, M-mode data 180, ARFI data 182, echocardiographic strain data 182, echocardiographic strain rate data 186 and tissue Doppler data 188, all of which may be stored in a memory 190 (or memory 114 or memory 122 shown in FIG. 1) temporarily before subsequent processing. The data 172-188 may be stored, for example, as sets of vector data values, where each set defines an individual ultrasound image frame. The vector data values are generally organized based on the polar coordinate system.

A scan converter sub-module 192 access and obtains from the memory 190 the vector data values associated with an image frame and converts the set of vector data values to Cartesian coordinates to generate an ultrasound image frame 194 formatted for display. The ultrasound image frames 194 generated by the scan converter module 192 may be provided back to the memory 190 for subsequent processing or may be provided to the memory 114 or the memory 122.

Once the scan converter sub-module 192 generates the ultrasound image frames 194 associated with, for example, the strain data, strain rate data, and the like, the image frames may be restored in the memory 190 or communicated over a bus 196 to a database (not shown), the memory 114, the memory 122 and/or to other processors, for example, the motion tracking module 126.

As an example, it may be desired to view functional ultrasound images or associated data (e.g., strain curves or traces) relating to echocardiographic functions in real-time on the display 118 (shown in FIG. 1). To do so, the scan converter sub-module 192 obtains strain or strain rate vector data sets for images stored in the memory 190. The vector data is interpolated where necessary and converted into an X,Y format for video display to produce ultrasound image frames. The scan converted ultrasound image frames are provided to a display controller (not shown) that may include a video processor that maps the video to a grayscale mapping for video display (e.g., 2D gray-scale projection). The grayscale map may represent a transfer function of the raw image data to displayed gray levels. Once the video data is mapped to the grayscale values, the display controller controls the display 118 (shown in FIG. 1), which may include one or more monitors or windows of the display, to display the image frame. The echocardiographic image displayed in the display 118 is produced from image frames of data in which each datum indicates the intensity or brightness of a respective pixel in the display. In this example, the displayed image represents muscle motion in a region of interest being imaged based on 2D tracking applied to, for example, a multi-plane image acquisition.

Referring again to FIG. 2, a 2D video processor sub-module 194 combines one or more of the frames generated from the different types of ultrasound information. For example, the 2D video processor sub-module 194 may combine a different image frames by mapping one type of data to a grayscale map and mapping the other type of data to a color map for video display. In the final displayed image, color pixel data may be superimposed on the grayscale pixel data to form a single multi-mode image frame 198 (e.g., functional image) that is again re-stored in the memory 190 or communicated over the bus 196. Successive frames of images may be stored as a cine loop in the memory 190 or memory 122 (shown in FIG. 1). The cine loop represents a first in, first out circular image buffer to capture image data that is displayed in real-time to the user. The user may freeze the cine loop by entering a freeze command at the user interface 124. The user interface 124 may include, for example, a keyboard and mouse and all other input controls associated with inputting information into the ultrasound system 100 (shown in FIG. 1).

A 3D processor sub-module 200 is also controlled by the user interface 124 and accesses the memory 190 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

The motion tracking module 126 is also controlled by the user interface 124 and accesses the memory 190 to obtain ultrasound information, and as described in more detail below, generates motion tracking information for display, which in some embodiments is displayed as a 2D projection having a grayscale pattern.

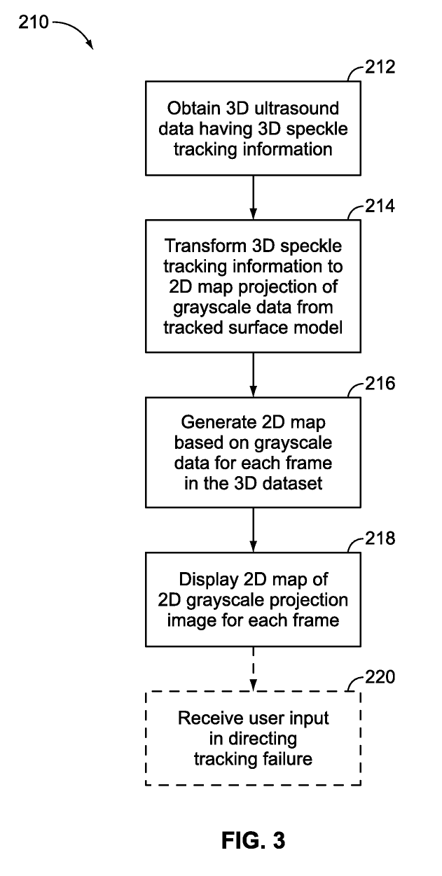

More particularly, a method 210 for generating tracking information from 3D ultrasound data is shown in FIG. 3. It should be noted that although the method 210 is described in connection with ultrasound imaging having particular characteristics, the various embodiments are not limited to ultrasound imaging or to any particular imaging characteristics. For example, although the method is described in connection with 3D speckle tracking, any type of motion tracking may be implemented. As another example, although the method is described in connection with a particular projection method to create the 2D projection, other projection methods may be implemented.

The method 210 includes obtaining 3D ultrasound data having 3D speckle tracking information at 212. For example, in some embodiments, the 3D ultrasound data includes ultrasound image information (e.g., image voxels) of an imaged object, such as a heart. Motion information is also obtained for the imaged object, which may be acquired using automatic tracking methods known in the art for tracking motion of tissue (e.g., myocardium) in 2D or 3D. For example, 3D speckle tracking may be used to track motion. The 3D ultrasound data may be stored data or currently acquired data. Additionally, the 3D data in various embodiments is a plurality of 2D or 3D datasets acquired over time. For example, in a cardiac application, the datasets may correspond to a plurality of images of an imaged heart over one or more heart cycles that form a 4D ultrasound dataset.

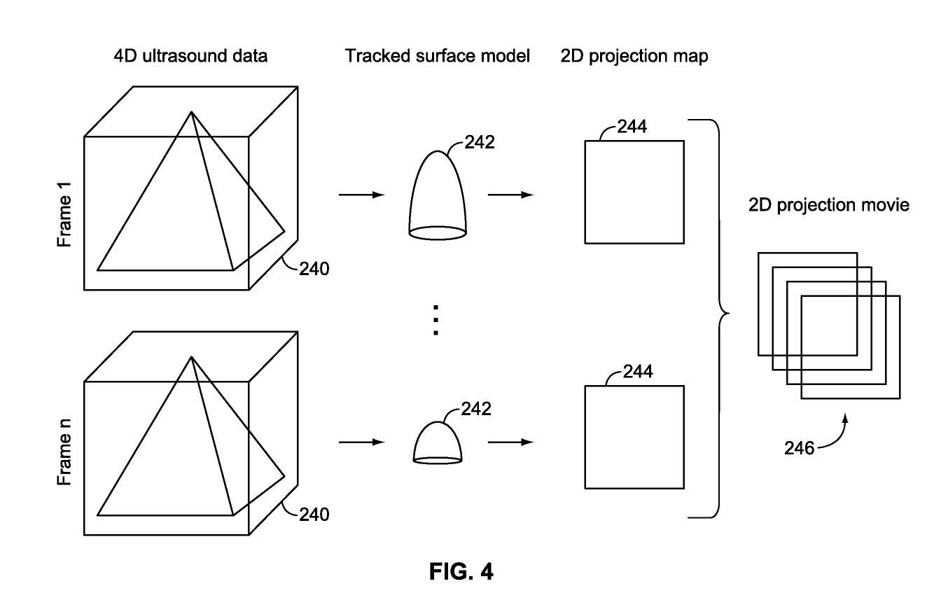

The acquired 3D ultrasound data with tracking information is then transformed to a 2D map projection at 214. The 2D map projection is a map projection of grayscale data from a tracked surface model. For example, in a cardiac application, the surface of the heart is represented as a model on a plane such that in any position of the surface model, a corresponding grayscale value from the 3D ultrasound data is determined with the 2D map projection based on the grayscale values at each location of the surface model (e.g., at each voxel that is projected onto a corresponding pixel). For example, as shown in FIG. 4, a plurality of frames 240 of 3D data (Frame 1 through Frame n are illustrated) forming a 4D ultrasound dataset (over time) are used to generate a tracked surface model 242 for each frame 240. The tracked surface models 242 correspond to an imaged heart in this example and show that the model changes in size as the imaged heart contracts and relaxes.

Referring again to the method 210 of FIG. 3, after the ultrasound tracking information as been transformed into 2D projections, a 2D map is generated at 216. The 2D map is based on the grayscale data (e.g., 3D grayscale speckle values) for each of the frames 240 (shown in FIG. 4) in the image dataset. Accordingly, as shown in FIG. 4, from the tracked surface models 242, a corresponding 2D projection map 244 is generated such that a 2D grayscale projection results for each frame 240 in the 4D ultrasound dataset. The grayscale value corresponding to each position on the surface model may be determined using an interpolation technique from the closest raw data samples. Alternatively, a larger (“thick surface”) region may be used, and a representative grayscale value from several samples in the radial direction of the region may be determined, for example, the maximum intensity along the radial direction.

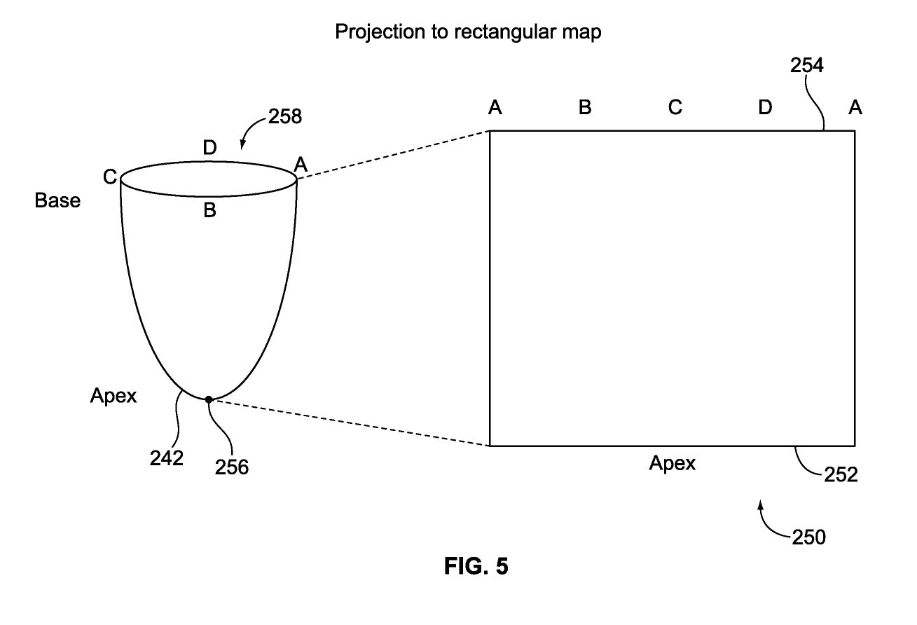

It should be noted that the 2D projection map 244 of the grayscale data may take different configurations or forms. For example, as shown in FIG. 5, the tracked surface model 242 is transformed to generate a rectangular map 250. The tracked surface model 242 is illustrated as a cardiac model such that one side 252 of the rectangular map 250 corresponds to an apex 256 of the tracked surface model and an opposite side 254 of the rectangular map 250 corresponds to a base 258 of the tracked surface model 242. Accordingly, grayscale motion information is projected from the tracked surface model 242 to the rectangular map 250 for each frame 240 of ultrasound data.

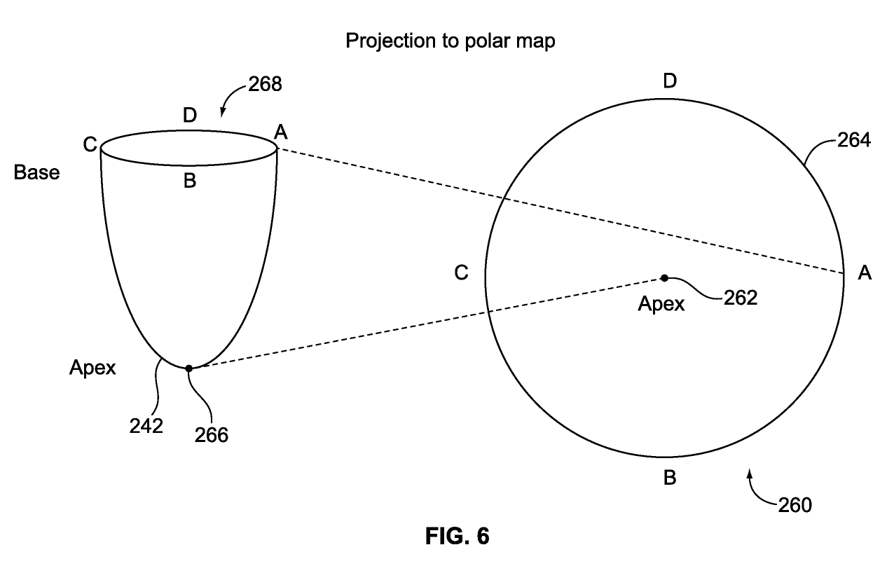

As another example, and as shown in FIG. 6, the tracked surface model 242 is transformed to generate a polar map 260 (illustrated as a circle). The tracked surface model 242 is illustrated as a cardiac model such that the center 262 of the polar map 260 corresponds to an apex 266 of the tracked surface model and a circumference 264 of the polar map 260 corresponds to a base 268 of the tracked surface model 242. Accordingly, grayscale motion information is projected from the tracked surface model 242 to the polar map 260 for each frame 240 of ultrasound data.

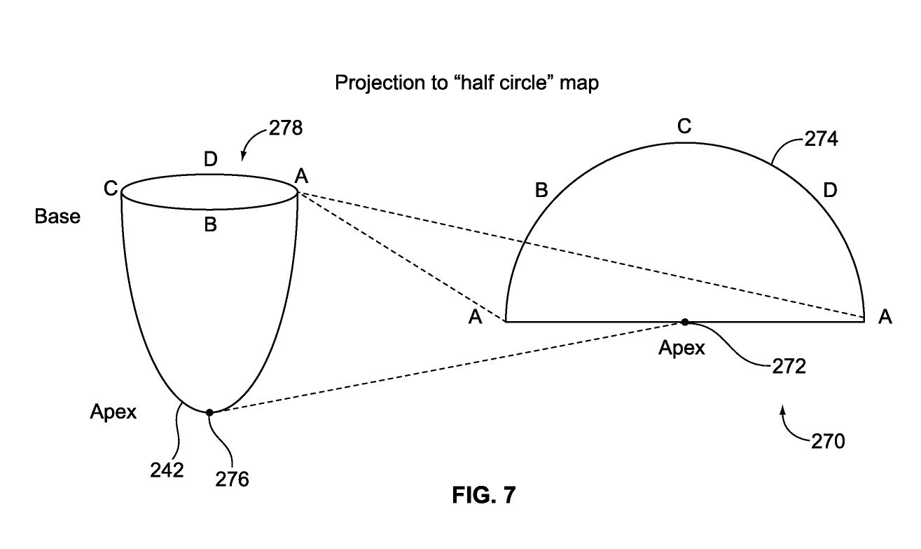

As still another example, and as shown in FIG. 7, the tracked surface model 242 is transformed to generate a semi-circle map 270 (a half circle). The tracked surface model 242 is illustrated as a cardiac model such that the center 272 of the semi-circle map 270 corresponds to an apex 276 of the tracked surface model and a circumference 274 of the semi-circle map 270 corresponds to a base 278 of the tracked surface model 242. Accordingly, grayscale motion information is projected from the tracked surface model 242 to the semi-circle map 270 for each frame 240 of ultrasound data.

It also should be noted that any map projection method may be used to create the 2D maps (or 2D projection images). However, in the various embodiments the 2D projection maps and the corresponding grayscale images therein are generated having the same size independent of the size and shape of the tracked surface model 242. For example, the grayscale image data within a 2D map corresponding to a tracked surface model 242 that is smaller in size than another tracked surface model 242 (e.g., a tracked surface model 242 corresponding to a contracted heart versus a relaxed heart) may be scaled using any scaling method such as increasing the size in the map portion corresponding to a particular point on the tracked surface model 242 or using extrapolation. Thus, in embodiments using 3D speckle tracking, when the tracking is good or acceptable, a substantially still speckle pattern is maintained over the entire 2D map.

Referring again to the method 210 of FIG. 3, after the 2D maps are generated, the 2D maps having the 2D grayscale projection images may be displayed for each frame at 218. For example, as shown in FIG. 4, the 2D projection maps 244 may be combined sequentially to form a 2D projection movie 246 (e.g., cine loop) corresponding to the 4D ultrasound data. Accordingly, the grayscale projection images are displayed sequentially such that if tracking is correct, ultrasound data from any tracked position of the tracked surface model 242 is displayed in a fixed position in the displayed 2D projection images within the 2D map. Thus, when motion tracking is good or acceptable, the 2D projection movie 246 of the 2D projection images displayed sequentially will appear as a substantially still image with no apparent motion in any direction (e.g., static grayscale pattern). If the tracking is less than good or not acceptable, indicating that the motion tracking has failed, the 2D projection movie 246 will show apparent motion (e.g., moving grayscale pattern). For example, motion may be apparent if the tracking occurred correctly perpendicular to the tracked surface model 242, but failed in the surface directions. As another example, motion may be apparent if the tracking failed perpendicular to the tracked surface model 242, which motion will be apparent out of plane motion in the 2D projection images displayed. Thus, poor or failed longitudinal or circumferential tracking are identified by movement in the 2D projection images.

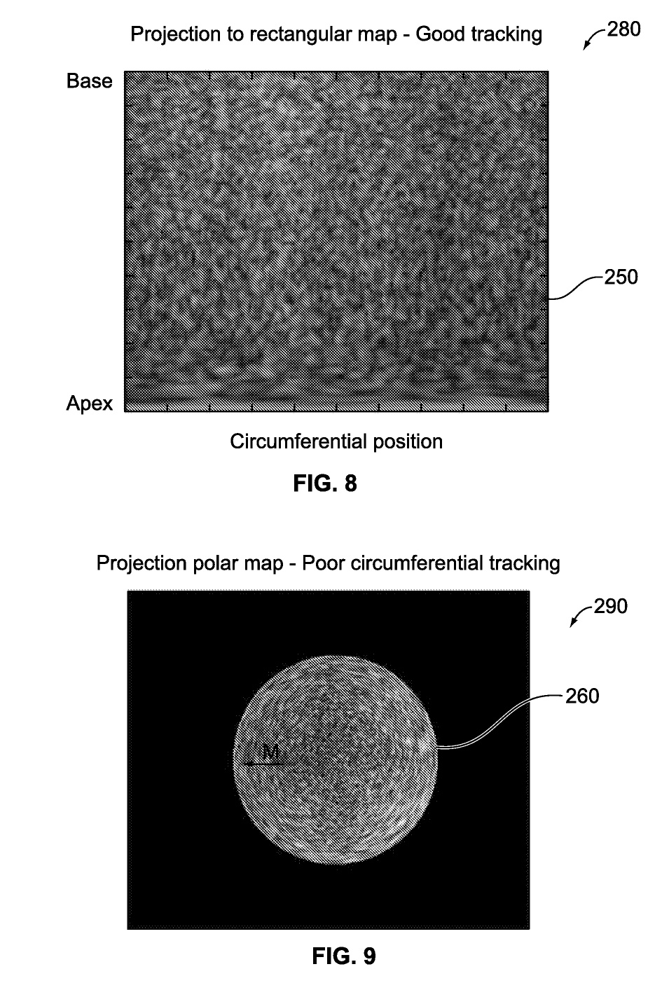

The various embodiments thereby generate 2D maps corresponding to 3D data and provide a display indicative of the quality of ultrasound motion tracking. For example, as shown in FIG. 8, a user interface 280, illustrated as display having the rectangular map 250 with a tracked motion display portion therein shows motion tracking grayscale data from a 3D dataset representing cardiac motion. As previously described herein, the top of the rectangular map 250 corresponds to the base of the tracked surface model of the heart and the bottom of the rectangular map 250 corresponds to the apex of the tracked surface model of the heart, with the horizontal axis corresponding to circumferential position along the tracked surface model. If the 2D projection image shown on the display 280 is a substantially static image or grayscale pattern, such that there is minimal or no apparent motion or no apparent motion in any direction, this static image condition is indicative of tracking quality that did not fail in any region, for example, tracking for all regions was good. However, motion within the 2D projection image or grayscale pattern is indicative of a potential issue or problem with the motion tracking. For example, apparent motion in a radial direction outward in the rectangular map 250 occurs if the motion tracking is not good because the grayscale pattern in every frame (at every time stamp) should be the same, but the pattern is different. The difference in the grayscale patterns produces the apparent motion in the displayed 2D projection image.

As another example, as shown in FIG. 9, a user interface 290, illustrated as a display having the polar map 260 with a tracked motion display portion therein shows motion tracking grayscale data from a 3D dataset representing cardiac motion. As previously described herein, the middle of the polar map 260 corresponds to the apex of the tracked surface model of the heart and the circumference of the polar map 260 corresponds to the base of the tracked surface model of the heart. If the 2D projection image shown on the display 280 is a substantially static image or grayscale pattern, such that there is minimal or no apparent motion, this static image condition is indicative of tracking quality that did not fail in any region, for example, tracking for all regions was good. However, motion within the 2D projection image is indicative of a potential issue or problem with the motion tracking. For example, apparent motion axially outward as illustrated by the arrow M (shown at 9 o'clock in the polar map 260 of FIG. 9) is indicative of poor or failed tracking in the corresponding region of the tracked surface model, namely poor longitudinal tracking. Apparent rotation in the 2D projection image is also indicative of poor or failed tracking in the corresponding region of the tracked surface model, namely poor circumferential tracking.

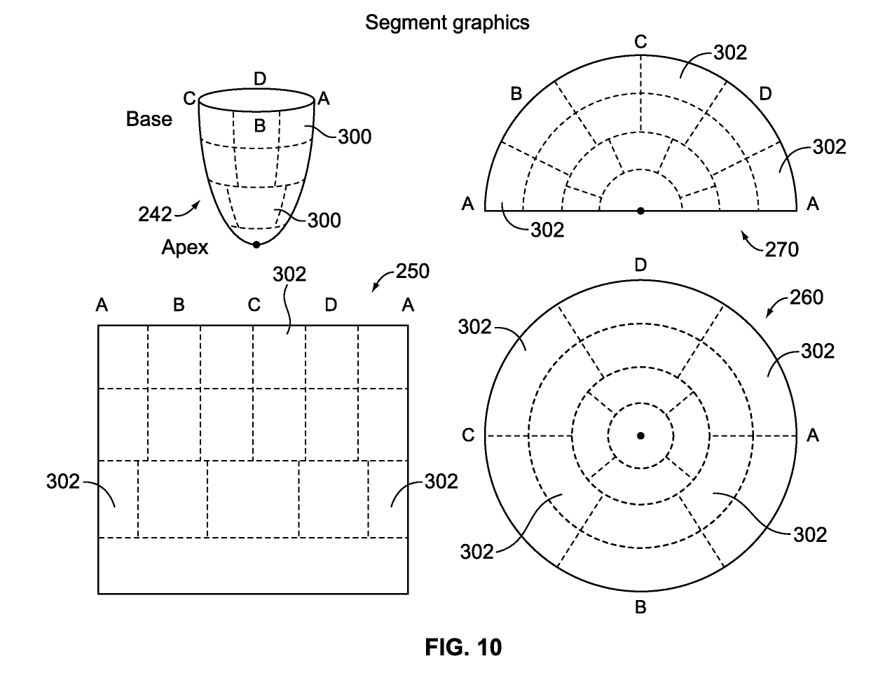

Accordingly, in the various embodiments, motion tracking quality may be assessed based on movement or non-movement of the grayscale 2D projection image or grayscale pattern within the 2D map. The amount of movement may be used to determine whether the tracking failed or was poor, for example, based on predetermined thresholds for the movement. Thus, a user is able to determine where and how the tracking failed based on the location of the movement and the type of movement, respectively. It should be noted that a graphic, for example, segment overlays or segment graphics may be displayed in combination with the 2D map as shown in FIG. 10. For example, the tracked surface model 242 may be divided into a plurality of segments 300. Each of the rectangular map 250, polar map 260 and semi-circle map 270 may include segments 302 corresponding to the segments 300 of the tracked surface model 242 to associate regions within the maps 250, 260 and 270 with regions of the tracked surface model 242. Thus, movement or motion within one of the segments 302 may be correlated with segment 300 of the tracked surface model 242 to determine the portion of the tracked surface model 242 where the quality of motion tracking may be poor or failed. Thus, for example, the polar map 260 is configured as a bull's eye plot.

Based on observed apparent motion, and referring again to the method 210 of FIG. 3, a user input indicating a tracking failure may be received at 220, which may be used to help processing by a tracking program or algorithm. For example, a user may click or drag that portion of the 2D projection image within the 2D map where there is apparent motion, for example, using a computer mouse. The user may, for example, deform or move that portion of the 2D projection image in an opposite direction and in amount about equal to the apparent motion to indicate where and in what direction the tracking failed. Thereafter, the tracking information may be updated accordingly or another tracking process performed.

It should be noted that other information or images may be displayed in combination with (e.g., concurrently with) or separate from the 2D map. For example, other tracking quality displays such as tracked or deformed 2D slices or M-mode or segmental renderings can be displayed (for the entire region or a sub-region selected by a user), which may be generated and displayed in any manner known in the art.

It also should be noted that the 2D projection images may be used for automatic analysis. For example, a 2D speckle tracking process may be used to eliminate residual motion based on the tracked motion. The 2D speckle tracking process may be used to improve the 3D tracking (e.g., correct poor tracking) or to add information to the 2D projection images, such as color coding the motion. As another example, additional information such as color coding of automatic estimated 3D tracking quality may be displayed (with different colors representing different levels of quality).

The various embodiments also may display information to facilitate visualizing poor tracking such as the additional displays described above. Other information can include, for example, color coding of the point projection of the radial vector or a radial tracking quality estimate. As another example, a thick slice semi-transparent 3D rendering may also be displayed to show radial direction data. In some embodiments the 2D projection movie may be presented as a 3D rendering. For example, the 2D projection images may be stacked to create a semi-transparent rendering to allow a user to check for straight lines in the motion tracking.

It should be noted that different post-processing procedures may be performed. For example, the 2D projection images may be post-processed to improve visualization, such as by using temporal filtering or histogram equalization to remove gross intensity changes.

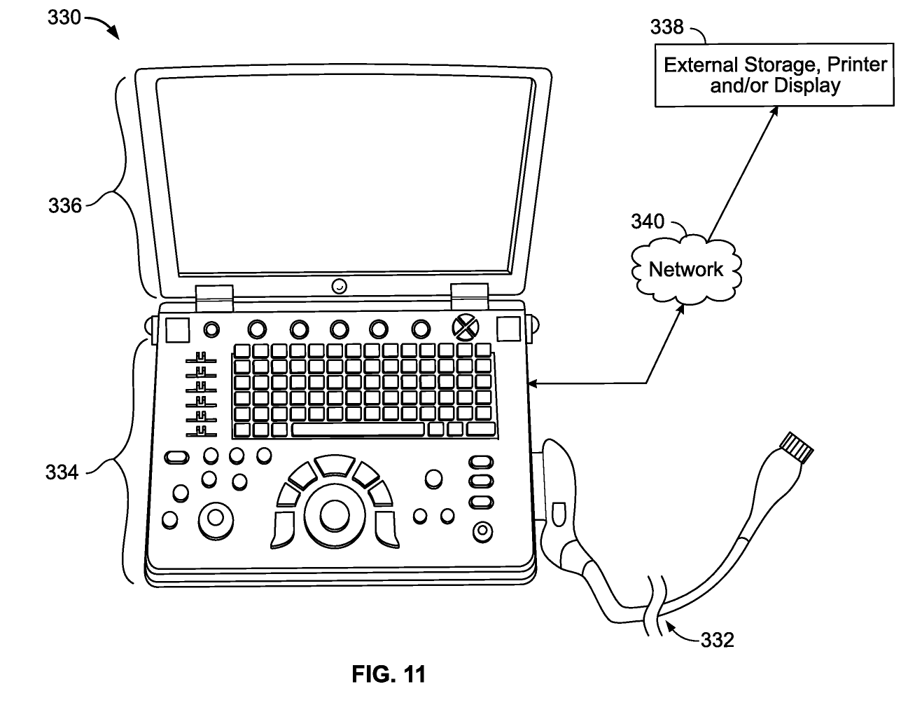

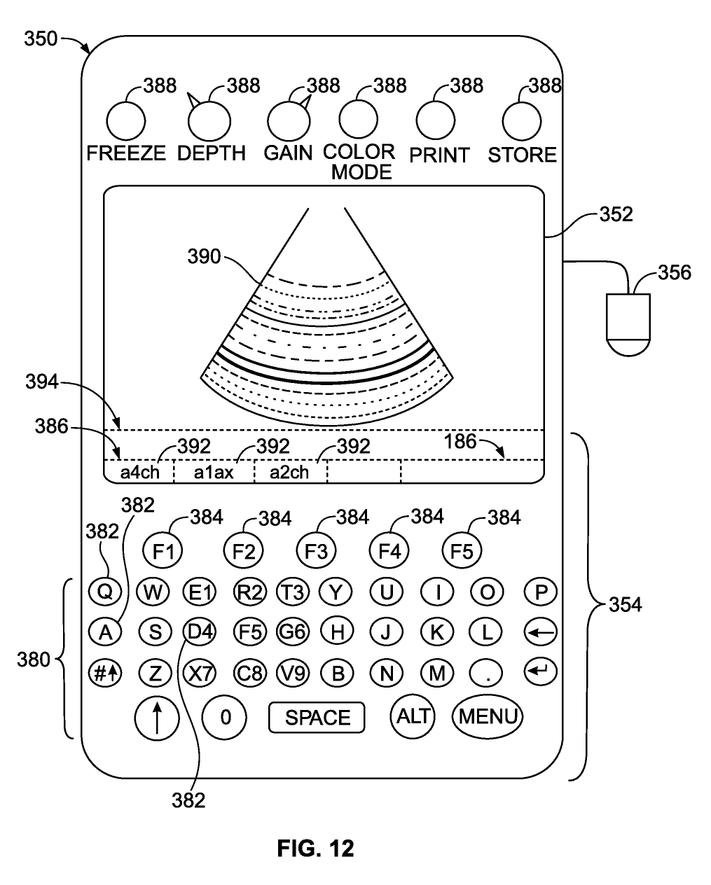

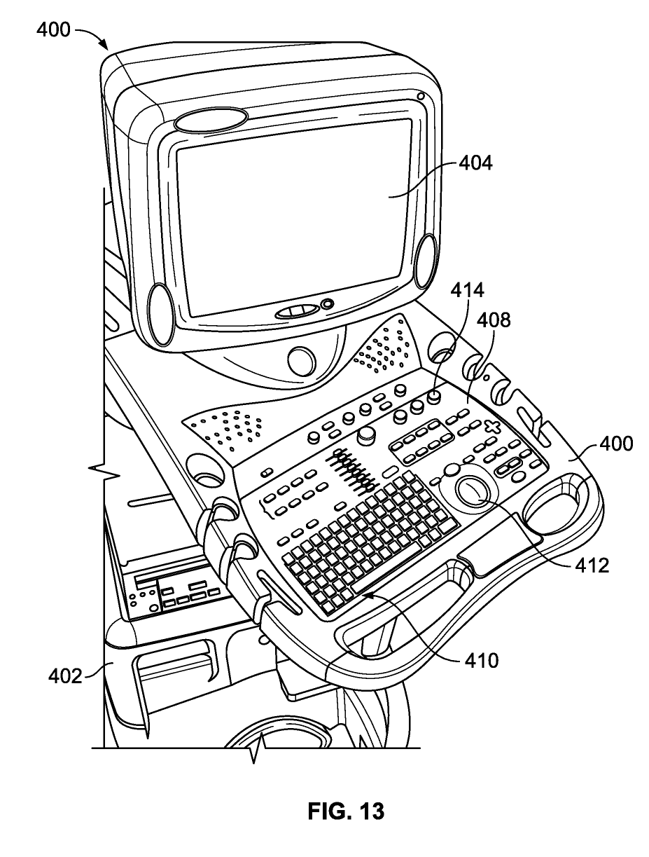

The ultrasound system 100 of FIG. 1 may be embodied in a small-sized system, such as laptop computer or pocket sized system as well as in a larger console-type system. FIGS. 11 and 12 illustrate small-sized systems, while FIG. 13 illustrates a larger system.

FIG. 11 illustrates a 3D-capable miniaturized ultrasound system 330 having a probe 332 that may be configured to acquire 3D ultrasonic data or multi-plane ultrasonic data. For example, the probe 332 may have a 2D array of elements 104 as discussed previously with respect to the probe 106 of FIG. 1. A user interface 334 (that may also include an integrated display 336) is provided to receive commands from an operator. As used herein, “miniaturized” means that the ultrasound system 330 is a handheld or hand-carried device or is configured to be carried in a person's hand, pocket, briefcase-sized case, or backpack. For example, the ultrasound system 330 may be a hand-carried device having a size of a typical laptop computer. The ultrasound system 330 is easily portable by the operator. The integrated display 336 (e.g., an internal display) is configured to display, for example, one or more medical images.

The ultrasonic data may be sent to an external device 338 via a wired or wireless network 340 (or direct connection, for example, via a serial or parallel cable or USB port). In some embodiments, the external device 338 may be a computer or a workstation having a display. Alternatively, the external device 338 may be a separate external display or a printer capable of receiving image data from the hand carried ultrasound system 330 and of displaying or printing images that may have greater resolution than the integrated display 336.

FIG. 12 illustrates a hand carried or pocket-sized ultrasound imaging system 350 wherein the display 352 and user interface 354 form a single unit. By way of example, the pocket-sized ultrasound imaging system 350 may be a pocket-sized or hand-sized ultrasound system approximately 2 inches wide, approximately 4 inches in length, and approximately 0.5 inches in depth and weighs less than 3 ounces. The pocket-sized ultrasound imaging system 350 generally includes the display 352, user interface 354, which may or may not include a keyboard-type interface and an input/output (I/O) port for connection to a scanning device, for example, an ultrasound probe 356. The display 352 may be, for example, a 320×320 pixel color LCD display (on which a medical image 190 may be displayed). A typewriter-like keyboard 380 of buttons 382 may optionally be included in the user interface 354.

Multi-function controls 384 may each be assigned functions in accordance with the mode of system operation (e.g., displaying different views). Therefore, each of the multi-function controls 384 may be configured to provide a plurality of different actions. Label display areas 386 associated with the multi-function controls 384 may be included as necessary on the display 352. The system 350 may also have additional keys and/or controls 388 for special purpose functions, which may include, but are not limited to “freeze,” “depth control,” “gain control,” “color-mode,” “print,” and “store.”

One or more of the label display areas 386 may include labels 392 to indicate the view being displayed or allow a user to select a different view of the imaged object to display. For example, the labels 392 may indicate an apical 4-chamber view (a4ch), an apical long axis view (alax) or an apical 2-chamber view (a2ch). The selection of different views also may be provided through the associated multi-function control 384. For example, the 4ch view may be selected using the multi-function control F5. The display 352 may also have a textual display area 394 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 350 and the miniaturized ultrasound system 330 may provide the same scanning and processing functionality as the system 100 (shown in FIG. 1).

FIG. 13 illustrates a portable ultrasound imaging system 400 provided on a movable base 402. The portable ultrasound imaging system 400 may also be referred to as a cart-based system. A display 404 and user interface 406 are provided and it should be understood that the display 404 may be separate or separable from the user interface 406. The user interface 406 may optionally be a touchscreen, allowing the operator to select options by touching displayed graphics, icons, and the like.

The user interface 406 also includes control buttons 408 that may be used to control the portable ultrasound imaging system 400 as desired or needed, and/or as typically provided. The user interface 406 provides multiple interface options that the user may physically manipulate to interact with ultrasound data and other data that may be displayed, as well as to input information and set and change scanning parameters and viewing angles, etc. For example, a keyboard 410, trackball 412 and/or multi-function controls 414 may be provided.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for providing ultrasound information, the method comprising:
obtaining three-dimensional (3D) ultrasound image data of a scanned object, the 3D ultrasound image data including motion tracking information;
transforming the 3D ultrasound image data with the motion tracking information to a two-dimensional (2D) map projection; and
generating a 2D map based on the 2D map projection.

2. A method in accordance with claim 1 wherein the 2D map projection maps each of a plurality of desired anatomical positions in the 3D ultrasound image data to a fixed 2D coordinates in the 2D map.

3. A method in accordance with claim 1 wherein the 2D map projection maps to a fixed size 2D map independent of a size of the scanned object in the 3D ultrasound image data.

4. A method in accordance with claim 1 wherein the motion tracking information includes grayscale data from a tracked surface model.

5. A method in accordance with claim 1 wherein the motion tracking information comprises 3D speckle tracking information.

6. A method in accordance with claim 1 wherein the 3D ultrasound image data includes a plurality of frames of 3D ultrasound data together forming a four-dimensional (4D) dataset and further comprising combining a plurality of 2D projection maps corresponding to each of the frames of 3D ultrasound data to generate a 2D projection movie.

7. A method in accordance with claim 6 wherein combining the plurality of projection maps comprises sequentially combining the projection maps.

8. A method in accordance with claim 6 further comprising displaying the 2D projection movie in a projection map display.

9. A method in accordance with claim 8 wherein the projection map display comprises one of a rectangular map, a polar map and a semi-circle map.

10. A method in accordance with claim 6 further comprising displaying the 2D projection movie wherein apparent motion within the 2D projection movie is indicative of a quality of motion tracking based on the motion tracking information.

11. A method in accordance with claim 10 wherein the apparent motion is indicative of a quality of one of longitudinal motion tracking and circumferential motion tracking.

12. A method in accordance with claim 10 further comprising receiving a user input indicating at least one of a location and an amount of a tracking failure.

13. A method in accordance with claim 1 wherein the object comprises a heart.

14. A method in accordance with claim 1 wherein the object comprises a heart and the 2D map projection comprises a map projection of grayscale data from a tracked surface model of the heart, and further comprising determining a grayscale value from the 3D ultrasound data with the 2D map projection based on the grayscale values at each location of the surface model.

15. A method in accordance with claim 1 further comprising displaying the 2D map with segments that correspond to the segments of a tracked surface model of the imaged object.

16. A user interface comprising:
a two-dimensional (2D) map portion corresponding to a tracked surface model of a three-dimensional (3D) ultrasound imaged object; and
a tracked motion display portion within the 2D map portion displaying grayscale motion information representative of tracked motion of the imaged object based on the tracked surface model.

17. A user interface in accordance with claim 16 wherein the grayscale motion information comprises 3D speckle tracking information.

18. A user interface in accordance with claim 16 wherein the tracked motion display portion displays a grayscale pattern wherein motion is indicative of failed tracking.

19. An ultrasound imaging system comprising:
an ultrasound probe configured to acquire three-dimensional (3D) images of an object; and
a processor having a motion tracking module configured to generate a two-dimensional (2D) map projection based on grayscale motion data from the 3D images.

20. An ultrasound imaging system in accordance with claim 19 further comprising a display configured to display sequentially the 2D map projection for a plurality of frames of the 3D images, wherein apparent motion in the displayed 2D map projections is indicative of a quality of tracking.

21. An ultrasound imaging system in accordance with claim 19 wherein the object comprises a heart.

22. An ultrasound imaging system in accordance with claim 19 further comprising a user interface configured to receive a user input indicating at least one of a location and an amount of a tracking failure.
